(19) United States
(12) Patent Application Publication
(10) Pub. No.: US 2009/0105528 A1
(43) Pub. Date: Apr. 23, 2009

(54) SEXUAL STIMULATION AID

(76) Inventor: David Seaman, Spring Hill, FL (US)

Correspondence Address: Maxey Law Offices, PLLC, 13630 58TH ST. NORTH, SUITE 101, CLEARWATER, FL, 33760, US

(21) Appl. No.: 12/177,308

(22) Filed: Jul. 22, 2008

Related U.S. Application Data
(60) Provisional application No. 60/981,945, filed on Oct. 23, 2007.

(51) Int. Cl. A61F 5/00

(52) U.S. Cl. 600/38

(57) ABSTRACT

A sexual stimulation aid with a phallus-shaped portion for insertion into a human cavity capable of receiving sexual pleasure. The phallus-shaped portion has a engaging surface engagable with a chin of a wearer such that the phallus-shaped portion extends in a direction generally forwardly and outwardly of the chin. A strap secures the phallus-shaped portion to a wearer with the engaging surface engaged with the chin of the wearer. An extension extends from the phallus-shaped portion and has at least one surface that is engagable with a body portion of a receiver of the phallus-shaped portion. An electrically powered vibrator unit is contained within the phallus-shaped portion to provide vibratory stimulus. A second vibration unit may be contained within the extension.

### CROSS-REFERENCE TO RELATED APPLICATIONS

This application claims the benefit of U.S. Provisional Application No. 60/981,945, filed Oct. 23, 2007, the entire of which is incorporated herein by reference.

### FIELD OF THE INVENTION

The present invention relates generally to devices and methods used for sexual pleasure, and more particularly, relating to an artificial phallus worn on user's head to provide stimulation to a recipient of the phallus.

### BACKGROUND OF THE INVENTION

There is a growing acceptance of sexuality, as exhibited by popular acceptance of the near-universality of masturbation, and the growth of the market for sexual devices. Prior to this shift there have been a plethora of devices sold for sexual pleasure, although primarily under the euphemistic names and a pretense of providing “massage”. Modern sexual devices fall broadly into two classes: mechanized and non-mechanized. Mechanized devices typically vibrate, although there are examples that rotate, thrust, and even circulate small beads within an elastomeric shell. Non-mechanized devices are made from a solid mass of rigid or semi-rigid material in a variety of shapes.

Although not spoken of openly in Western society until recently, oral sex is accorded a revered place in many cultures. Oral sex is the act of using the mouth, lips, and tongue to stimulate the female genitals. Oral sex is often accompanied by the insertion of finger(s) or a sexual aid into the vagina and/or into the anus, which allows for the simultaneous stimulation of the recipients sexual pleasure zones, which many women find can produce very intense sensations.

What is desirable is a device that can be worn by a sex partner while performing oral sex that provides enhanced stimulation and sensation to various sexual pleasure zones of the recipient simultaneously without requiring involvement of the providers hands.

### SUMMARY OF THE INVENTION

The preferred embodiments of the present invention addresses this need by providing a phallus-shaped device that is attachable to a user's head such that the phallus-shaped device can be received by a body cavity capable of receiving sexual pleasure of the recipient while the user performs oral sex on the recipient and which is attachable to the user's head by a strap, thereby freeing the user's hands from holding the phallus-shaped device.

To achieve these and other advantages, in general, in one aspect, device for sexual pleasure is provided. The device includes a phallus-shaped portion for insertion into a human cavity capable of receiving sexual pleasure, the phallus-shaped portion having a engaging surface engagable with a chin of a wearer such that the phallus-shaped portion extends in a direction generally forwardly and outwardly of the chin, a means for securing the phallus-shaped portion to a wearer with the engaging surface engaged with the chin of the wearer, an extension extending from the phallus-shaped portion, the extension having at least one surface that is engagable with a body portion of a receiver of said phallus-shaped portion, a vibrator unit contained within the phallus-shaped portion, a power source, and a controller operatively connecting the power source to the vibrator.

In general, in another aspect, the device may further include a second vibrator unit contained within the extension, the controller operatively connecting the power source to the second vibrator.

In general, in yet another aspect, the extension extends in a forwardly direction and terminates at a end that is laterally spaced from the phallus-shaped portion.

In general, in another aspect, the extension is insertable into a human cavity capable of receiving sexual pleasure.

In general, in another aspect, the phallus-shaped portion has a peripheral exterior wall surface, and further wherein the engaging surface is bounded by the peripheral exterior wall surface.

In general, in yet another aspect, the extension terminates at a location intermediated a forward end of the phallus-shaped portion and the rearward end of the phallus-shaped portion.

There has thus been outlined, rather broadly, the more important features of the invention in order that the detailed description thereof that follows may be better understood and in order that the present contribution to the art may be better appreciated.

Numerous objects, features and advantages of the present invention will be readily apparent to those of ordinary skill in the art upon a reading of the following detailed description of presently preferred, but nonetheless illustrative, embodiments of the present invention when taken in conjunction with the accompanying drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

The accompanying drawings, which are included to provide further understanding of the invention and are incorporated in and constitute a part of this specification, illustrate preferred embodiments of the invention and together with the description serve to explain the principles of the invention, in which:

FIG. 1 is a diagrammatic side view of the sexual stimulation aid constructed in accordance with the principles of the present invention;

FIG. 2 is a diagrammatic top view of the sexual stimulation aid;

FIG. 3 is a diagrammatic partial review view of the sexual stimulation aid; and

FIG. 4 is a diagrammatic partial cross-section view of the sexual stimulation aid showing a vibration unit.

### DETAILED DESCRIPTION OF THE INVENTION

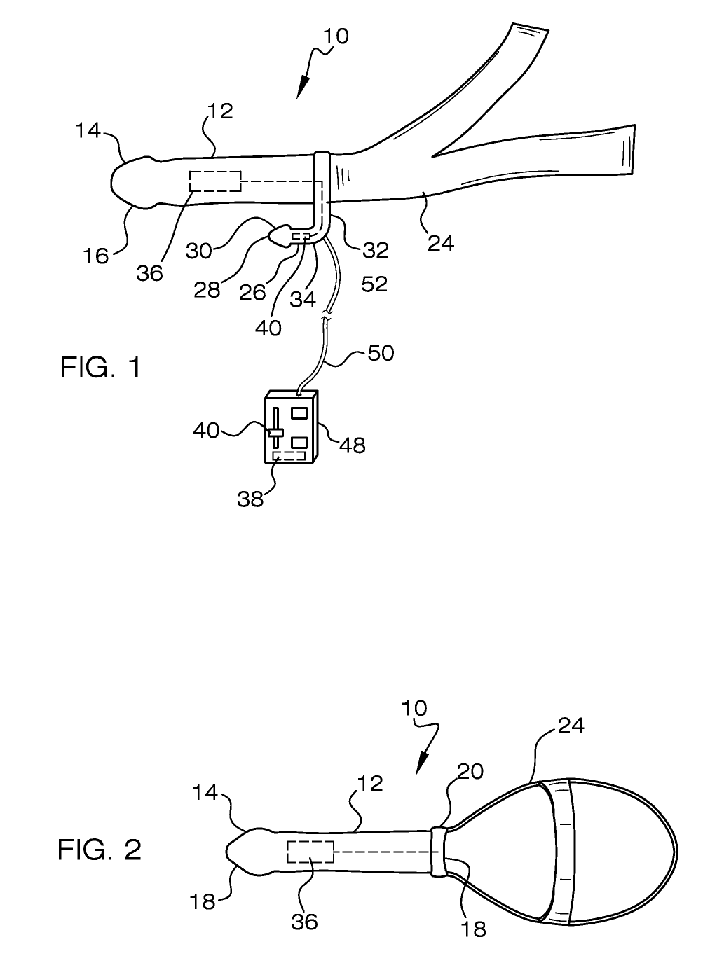

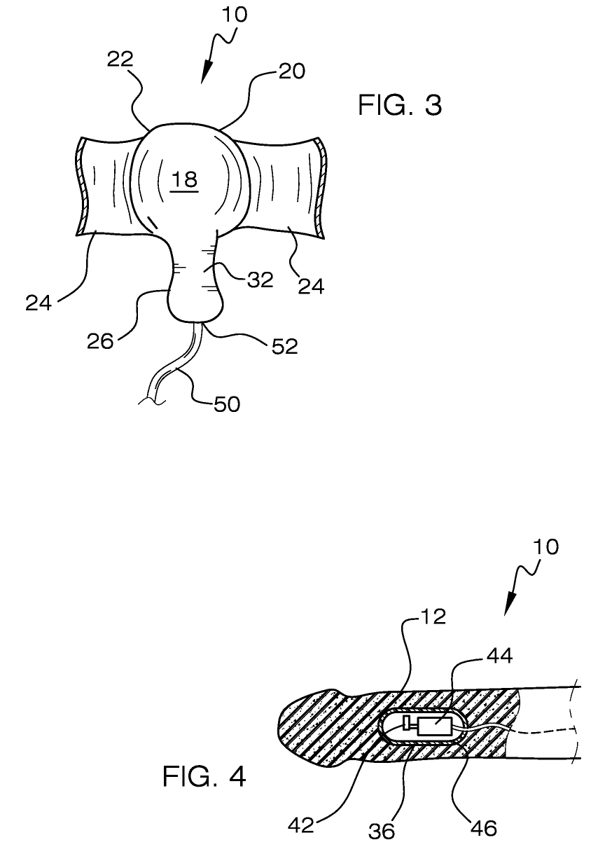

Referring now to FIGS. 1-4 of the drawings, reference numeral 10 generally designates the sexual stimulation aid of the present invention. The sexual stimulation aid 10 is configured to be worn on the head of a user for use while performing oral sex to simultaneously stimulate other sexual pleasure zones of the recipient.

In the preferred embodiment, the sexual stimulation aid 10 includes a phallus-shaped portion 12 configured and shaped to resemble the look and feel of an erect penis. The phallus-shaped portion 12 is insertable into a human cavity capable of receiving sexual pleasure, such as the vagina of a woman. The phallus-shaped portion 12 would be inserted into the vagina while the wearer performs oral sex on the recipient to enhance and increase the sexual pleasure of the recipient.

The phallus-shaped portion 12 can be made of any semi-rigid yet soft material, such as rubber-like, silicone, vinyl, the like. The phallus-shaped portion 12 may have a bulbous head 14 defined by a forward end 16 thereof. A chin engaging surface 18 is defined by a rearward end 20 of the phallus-shaped portion. The chin engaging surface 18 is bounded by the peripheral sidewall 22 of the phallus-shaped portion 12. The chin engaging surface 18 may be padded to provide comfort to the wearer.

A head strap 24 secures the sexual stimulation aid 10 to the head of a user with the chin engaging surface engaged with the chin of the user, and such that the phallus-shaped portion 12 extends in a direction generally forwardly and outwardly of the chin. The head strap 24 may be of an elastomeric material which stretches to conform to the size and shape of the wearer's head ensuring a secure attachment without requiring the adjustment of buckles.

An extension 26 for simultaneously stimulating the clitoris or the anus of the recipient extends outwardly from the phallus-shaped portion 12 at a location towards the rearward end 20 thereof. At least one surface 28 is engagable with a body portion of the receiver for stimulating the body portion. The extension 26 may extend in a forwardly direction and terminate at a end 30 that is laterally spaced from said phallus-shaped portion 12. The extension 26 may include a first portion 32 that is generally lateral to said phallus-shaped portion 12, and a second portion 34 that is generally parallel to said phallus-shaped portion. The extension 26 may terminate at a location intermediated the forward end 16 and the rearward 20 end of said phallus-shaped portion 12. Further, the extension 26 may be phallus-shaped.

The sexual stimulation aid 10 may further include vibration unit 36 positioned and contained within the phallus-shaped portion 12 for providing a vibratory stimulation to the recipient. The vibration unit 36 is well known in the art, and includes a spinner 42 driven by a motor 44. Both the spinner 42 and motor 44 are enclosed in a housing 46. A power source 38 is connected to the vibration unit 36 through a controller 40 to provide electrical power to the vibration unit causing the motor 44 to spin the spinner 42 in an offset fashion such that the entire vibration unit 36 vibrates. A second vibration unit 40 may be positioned and contained within the extension 26. The second vibration unit 40 is also connected to the power source 38 by the controller 40. The second vibration unit 40 is of the same construction has the first vibration unit 36. Alternatively, a solid state vibration device, known in the art, could be substituted in place of the motor driven spinner vibration unit.

The controller 40 and power source 38 are contained in a housing 48 that is remote from the phallus-shaped portion 12. The vibration units 36 and 40 are connected to the power source 38 via the controller by a cable 50. The cable 50 runs internally through the phallus-shaped portion 12 and extension 26 where it exists at a bottom portion 52 of the extension at a position just forward of the chin engaging surface 18 so as not to interfere with the chin of a user.

A number of embodiments of the present invention have been described. Nevertheless, it will be understood that various modifications may be made without departing from the spirit and scope of the invention. Accordingly, other embodiments are within the scope of the following claims.

What is claimed is:

1. A sexual stimulation device, comprising:
a phallus-shaped portion for insertion into a human cavity capable of receiving sexual pleasure, said phallus-shaped portion having a engaging surface engagable with a chin of a wearer such that said phallus-shaped portion extends in a direction generally forwardly and outwardly of the chin;
a means for securing said phallus-shaped portion to a wearer with said engaging surface engaged with the chin of the wearer;
an extension extending from said phallus-shaped portion, said extension having at least one surface that is engagable with a body portion of a receiver of said phallus-shaped portion;
a vibrator unit contained within said phallus-shaped portion;
a power source; and
a controller operatively connecting said power source to said vibrator.

2. The device of claim 1, further comprising:
a second vibrator unit contained within said extension, said controller operatively connecting said power source to said second vibrator.

3. The device of claim 1, wherein said extension extends in a forwardly direction and terminates at a end that is laterally spaced from said phallus-shaped portion.

4. The device of claim 3, wherein said extension is insertable into a human cavity capable of receiving sexual pleasure.

5. The device of claim 3, further comprising:
a second vibrator unit contained within said shaft, said controller operatively connecting said power source to said second vibrator.

6. The device of claim 1, wherein said engaging surface is defined by a rearward end of said phallus-shaped portion.

7. The device of claim 1, wherein said a means for securing said phallus-shaped portion to a wearer with said engaging surface engaged with the chin of the wearer comprises a head strap.

10. A sexual stimulation device, comprising:
a phallus-shaped portion for insertion into a human cavity capable of receiving sexual pleasure, said phallus-shaped portion having a engaging defined by a rearward end thereof, said engaging surface engagable with a chin of a wearer such that said phallus-shaped portion extends in a direction generally forwardly and outwardly of the chin;
a head strap for securing said phallus-shaped portion to the wear's head with said engaging surface engaged with the chin of the wearer;
an extension extending from said phallus-shaped portion in a forwardly direction and terminating at a end that is laterally spaced from said phallus-shaped portion, said extension having at least one surface that is engagable with a body portion of a receiver of said phallus-shaped portion, said extension being insertable into a human cavity capable of receiving sexual pleasure;
a first vibrator unit contained within said phallus-shaped portion;
a second vibrator unit contained with said extension;
a power source; and
a controller operatively connecting said power source to each of said first and said second vibrator units.

11. The device of claim 10, wherein said phallus-shaped portion has a peripheral exterior wall surface, and further wherein said engaging surface is bounded by said peripheral exterior wall surface.

12. The device of claim 10, further comprising:
a housing, said housing containing said controller and said power source.

13. The device of claim 10, wherein said extension is phallus-shaped.

14. The device of claim 10, wherein said extension terminates at a location intermediated a forward end of said phallus-shaped portion and said rearward end of said phallus-shaped portion.

15. The device of claim 10, wherein said extension includes a first portion that is generally lateral to said phallus-shaped portion, and a second portion that is generally parallel to said phallus-shaped portion.
